(19) United States
(12) Patent Application Publication
(10) Pub. No.: US 2023/0157688 A1
(43) Pub. Date: May 25, 2023

(54) ANVIL ASSEMBLY WITH CUTTING PLATE

(71) Applicant: Covidien LP, Mansfield, MA (US)

(72) Inventors: Zhinan Guo, Shanghai (CN); Ping Ren, Shanghai (CN)

(21) Appl. No.: 17/917,718

(22) PCT Filed: Apr. 9, 2020
(86) PCT No.: PCT/CN2020/083877

§ 371 Date: Oct. 7, 2022

(51) Int. Cl. A61B 17/072

(52) U.S. Cl. CPC A61B 17/072; A61B 2017/07257; A61B 2017/07285; A61B 2017/07221

(57) ABSTRACT

A surgical stapling device includes an end effector for a transverse type surgical stapling device that includes a U-shaped frame, an anvil assembly, and a cartridge assembly. The anvil assembly is supported on the U-shaped frame and includes a cutting plate that has a thickness that changes between its first and second ends to compensate for deformation of the frame during firing of the stapling device.

### FIELD

The present technology is generally related to surgical stapling devices and, more particularly, to surgical stapling devices that have a cutting knife and an anvil cutting plate to receive the cutting knife.

### BACKGROUND

Surgical stapling devices are commonly used during a variety of surgical procedures to expedite dissection and suturing of tissue and minimize trauma to a patient. Typically, the stapling devices include an end effector that includes a cartridge assembly and an anvil assembly. The cartridge assembly includes a knife assembly for cutting tissue and the anvil assembly supports a cutting plate which is engaged by the knife assembly to provide a more effective cutting process. The knife assembly includes a knife blade that has a sharpened cutting edge which is driven through tissue and into the cutting plate to transect or resect the tissue.

Surgical stapling devices are available in a variety of types for performing a variety of different surgical procedures. One type of stapling device includes a U-shaped end effector that has a first transverse portion, a second transverse portion, and a longitudinal portion interconnecting the first transverse portion and the second transverse portion. Each of the first and second end portions has a first end coupled to the longitudinal portion and a second free end. The anvil assembly is supported on the first transverse portion and the cartridge assembly is supported adjacent the second transverse portion and is movable towards the first transverse portion to move the anvil and cartridge assemblies into juxtaposed alignment.

During a surgical procedure, when the knife assembly is advanced into engagement the cutting plate of the anvil assembly, the free end of the first transverse portion supporting the anvil assembly tends to bend distally when the force of the knife assembly is applied to the anvil assembly. This causes the anvil assembly to bend distally such that the knife assembly applies a greater force on tissue nearer to the first end of the anvil assembly than to tissue nearer to the free end of the anvil assembly. This may result in ineffective cutting of tissue near the free end of the anvil assembly.

A continuing need exists in the art for an end effector that can compensate for distal deflection of the free end of the anvil assembly during cutting of tissue.

### SUMMARY

Aspects of this disclosure are directed to an end effector for a transverse type surgical stapling device that includes an end effector including a U-shaped frame, an anvil assembly, and a cartridge assembly. The U-shaped frame includes a first transverse portion, a second transverse portion, and a longitudinal portion that interconnects the first and second transverse portions. The first transverse portion includes a first end that is coupled to the longitudinal portion and a second free end. The anvil assembly is supported on the first transverse portion and includes a cutting plate and an anvil plate that is secured to the first transverse portion such that the cutting plate is sandwiched between the first transverse portion and the anvil plate. The cutting plate has a thickness that changes between its first and second ends to compensate for deformation of the first transverse portion of the frame during firing of the stapling device.

One aspect of the disclosure is directed to an end effector that includes a frame and an anvil assembly. The frame has a U-shaped configuration and includes a first transverse portion, a second transverse portion, and a longitudinal portion that interconnects the first transverse portion and the second transverse portion. The first transverse portion is spaced from the second transverse portion to define a gap. The first transverse portion of the frame has a first end coupled to the longitudinal portion of the frame and a second free end. The anvil assembly is supported on the first transverse portion of the frame and includes a cutting plate and an anvil plate. The cutting plate has a first end positioned adjacent the first end of the first transverse portion of the frame and a second end positioned adjacent the second free end of the first transverse portion of the frame. The anvil plate is secured to the first transverse portion of the frame proximally of the cutting plate such that the cutting plate is sandwiched between the anvil plate and the first transverse portion. The thickness of the cutting plate increases from the first end of the cutting plate to the second end of the cutting plate.

In aspects of the disclosure, the cutting plate is formed from a material selected from a polyether ether ketone, a polyoxymethylene, a polyphenylsulfone, a metal, polypropylene, polyethylene, a thermoplastic elastomer, polyurethane, thermoplastic polyurethanes or combinations thereof.

In some aspects of the disclosure, the cutting plate includes a proximally extending central rib and the anvil plate defines a knife slot that is aligned with the central rib.

In certain aspects of the disclosure, the cutting plate includes a body having a proximally facing surface, and the central rib extends proximally from the proximally facing surface.

In aspects of the disclosure, the proximally facing surface of the cutting plate includes first relief grooves that extend along opposite sides of the central rib of the cutting plate.

In some aspects of the disclosure, the cutting plate includes a distally facing surface that defines a second relief groove that is aligned with the central rib.

In certain aspects of the disclosure, the second relief groove has a width that extends across the central rib and the first relief grooves.

In aspects of the disclosure, the cutting plate has a side wall that extends about the body and extends proximally of the body to define a proximal recess and distally of the body to define a distal recess.

In some aspects of the disclosure, the first transverse portion of the frame is received within the distal recess and the anvil plate is received within the proximal recess.

In certain aspects of the disclosure, the anvil assembly is curved.

Another aspect of this discourse is directed to a surgical stapling device that includes a handle assembly, a frame, a clamp slide assembly, a cartridge assembly, and an anvil assembly. The frame defines a longitudinal axis and includes a distal frame portion having a U-shaped configuration. The distal frame portion includes a first transverse portion, a second transverse portion, and a longitudinal portion interconnecting the first transverse portion and the second transverse portion. The first transverse portion is spaced from the second transverse portion to define a gap. The first transverse portion of the distal frame portion has a first end coupled to the longitudinal portion of the distal frame portion and a second free end. The clamp slide assembly has a distal portion supported within the gap that includes a cartridge receiving portion. The cartridge assembly is supported on the cartridge receiving portion of the clamp slide assembly. The anvil assembly is supported on the first transverse portion of the distal frame portion and a cutting plate and an anvil plate. The cutting plate has a first end positioned adjacent the first end of the first transverse portion of the distal frame portion and a second end positioned adjacent the second free end of the first transverse portion of the distal frame portion. The anvil plate is secured to the first transverse portion of the distal frame portion proximally of the cutting plate such that the cutting plate is sandwiched between the anvil plate and the first transverse portion of the distal frame portion. The thickness of the cutting plate increases from the first end of the cutting plate to the second end of the cutting plate.

Another aspect of this disclosure is directed to an anvil assembly including a cutting plate and an anvil plate. The cutting plate has a first end and a second end. The anvil plate is positioned in juxtaposed alignment with the cutting plate. The thickness of the cutting plate increases from the first end of the cutting plate to the second end of the cutting plate.

Other features of the disclosure will be appreciated from the following description.

### BRIEF DESCRIPTION OF THE DRAWINGS

Various aspects of the disclosure are described herein below with reference to the drawings, wherein:

FIG. 1 is a side perspective view of a surgical stapling device including aspects of the disclosure;

FIG. 2 is a side perspective cutaway view of a distal portion of end effector of the surgical stapling device shown in FIG. 1 with parts separated;

FIG. 3 is a side perspective view of a cutting plate of an anvil assembly of the surgical stapling device shown in FIG. 1;

FIG. 4 is a cross-sectional view taken along section line 4-4 of FIG. 3;

FIG. 5 is a cross-sectional view taken along section line 5-5 of FIG. 3;

FIG. 6 is a cross-sectional view taken along section line 6-6 of FIG. 1;

FIG. 7 is an enlarged view of the indicated area of detail shown in FIG. 6;

FIG. 8 is a schematical cross-sectional view taken along section line 8-8 of FIG. 1;

FIG. 9 is a side perspective view of the end effector of the surgical stapling device shown in FIG. 1 in a clamped and fired position;

FIG. 10 is a cross-sectional view taken along section line 10-10 of FIG. 9;

FIG. 11 is an enlarged view of the indicated area of detail shown in FIG. 10; and

FIG. 12 is a side cutaway view of the end effector of the shown in FIG. 9 in the clamped and fired position with a knife assembly of a cartridge assembly and a thrust bar shown in phantom.

### DETAILED DESCRIPTION

The disclosed surgical stapling devices will now be described in detail with reference to the drawings in which like reference numerals designate identical or corresponding elements in each of the several views. However, it is to be understood that the aspects of the disclosure are merely exemplary and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. In addition, directional terms such as front, rear, upper, lower, top, bottom, distal, proximal, and similar terms are used to assist in understanding the description and are not intended to limit the disclosure.

In this description, the term “proximal” is used generally to refer to that portion of the device that is closer to a clinician during use of the device in its customary manner, while the term “distal” is used generally to refer to that portion of the device that is farther from the clinician during use of the device in its customary manner. In addition, the term “clinician” is used generally to refer to medical personnel including doctors, nurses, and support personnel. “About” or “approximately” or “substantially” as used herein may be inclusive of the stated value and means within an acceptable range of variation for the particular value as determined by one of ordinary skill in the art, considering the measurement in question and the error associated with measurement of the particular quantity (e.g., the limitations of the measurement system). For example, “about” may mean within one or more standard variations, or within ±10% of the stated value.

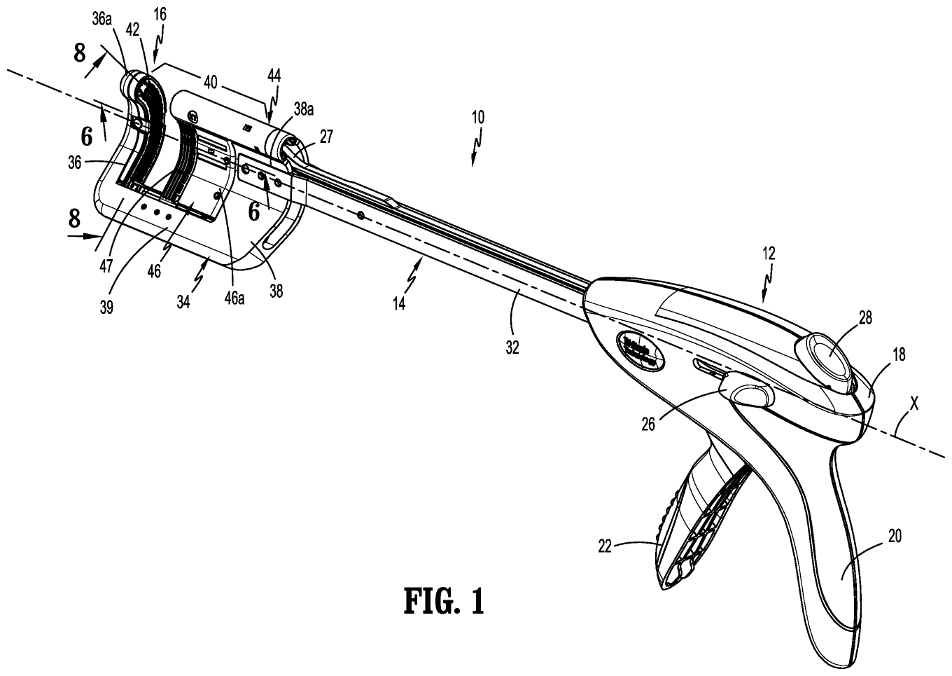

FIG. 1 illustrates the disclosed surgical stapling device shown generally as stapling device 10. The stapling device 10 includes a handle assembly 12, an elongate body 14 that extends distally from the handle assembly 12, and an end effector 16 that is supported on a distal portion of the elongate body 14. The elongate body 14 defines a longitudinal axis “X”. The handle assembly 12 includes a housing 18 that defines a stationary handle 20 and supports a movable trigger 22. In aspects of the disclosure, the movable trigger 22 is supported by the housing 18 to pivot towards the stationary handle 20 between non-actuated and actuated positions to operate the end effector 16. The handle assembly 12 also supports buttons 26 (only one is shown) that are positioned on each side of the housing 18 and are movable along the housing 18 to advance and retract an alignment pin pusher 27. The alignment pin pusher 27 is positioned and configured to engage an alignment pin (not shown) within the end effector to move the alignment pin between retracted position and advanced positions. The handle assembly 12 also includes a release button 28 that can be depressed to move the end effector 16 from a clamped position to an unclamped position. For a more detailed description of a suitable handle assembly 12, see, e.g., U.S. Pat. No. 6,817,508 (“the '508 Patent”).

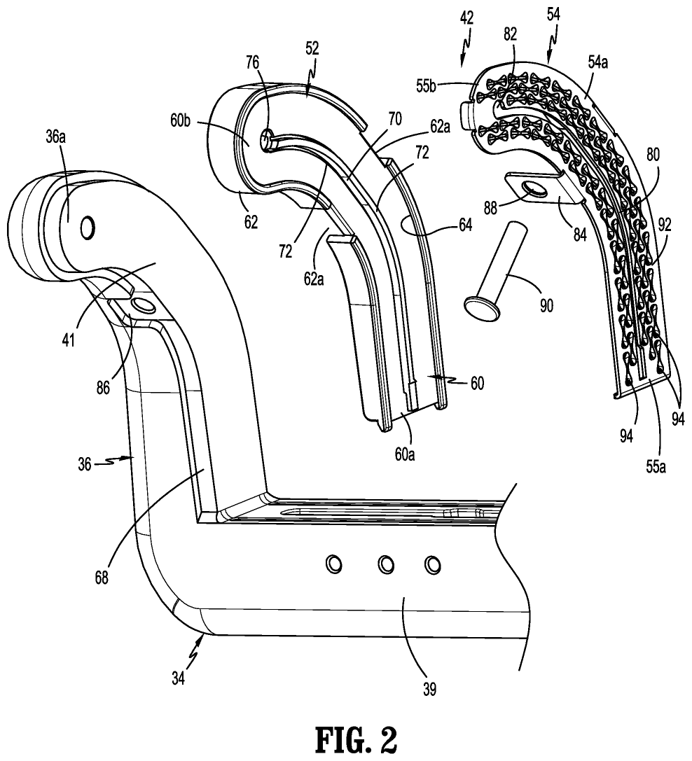

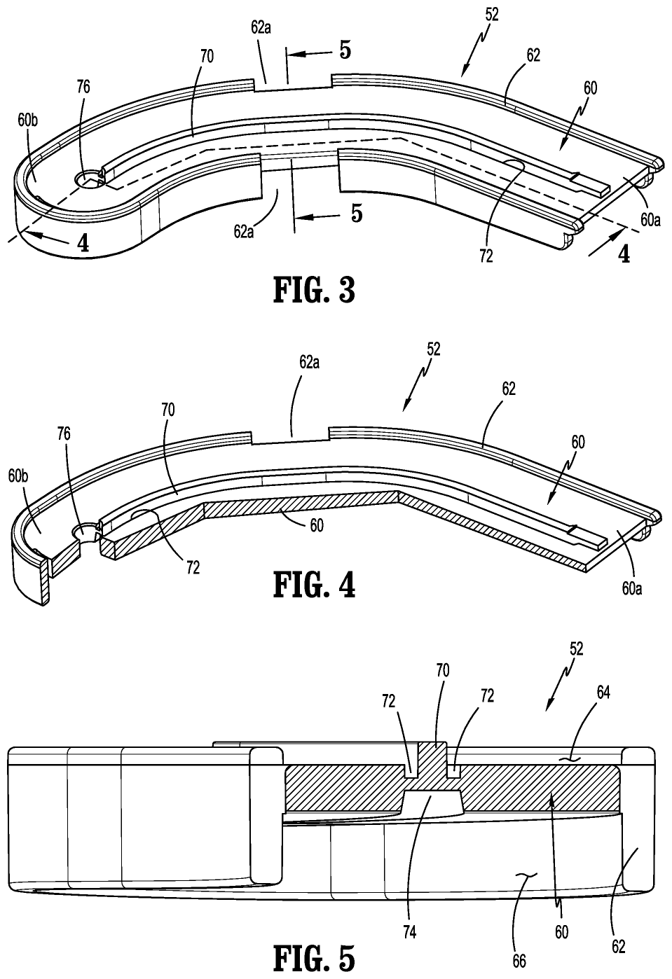

The stapling device 10 includes a frame 32 that extends from the handle assembly 12 to the end effector 16. The frame 32 includes a distal frame portion 34 that has a U-shaped configuration that forms a portion of the end effector 16. The distal frame portion 34 (FIG. 3) has a first transverse portion 36, a second transverse portion 38, and a longitudinal portion 39 that interconnects the first transverse portion 36 and the second transverse portion 38. The first and second transverse portions 36 and 38 are spaced from each other to define a gap 40 that extends between the first and second transverse portions 36 and 38. In some aspects of the disclosure, the first and second transverse portion 36 and 38 are curved along axes transverse to the longitudinal axis “X” of the elongate body 14 of the stapling device 10. Alternately, the first and second transverse portions may be linear or comprised of a plurality of linear portions that are positioned at angles in relation to each other. Each of the first and second transverse portions 36 and 38 has a first end coupled to (e.g., formed with) the longitudinal portion 39 of the distal frame portion 34 and a second free end 36a and 38a. The first transverse portion 36 has a substantially flat or planar proximal surface 41 (FIG. 2).

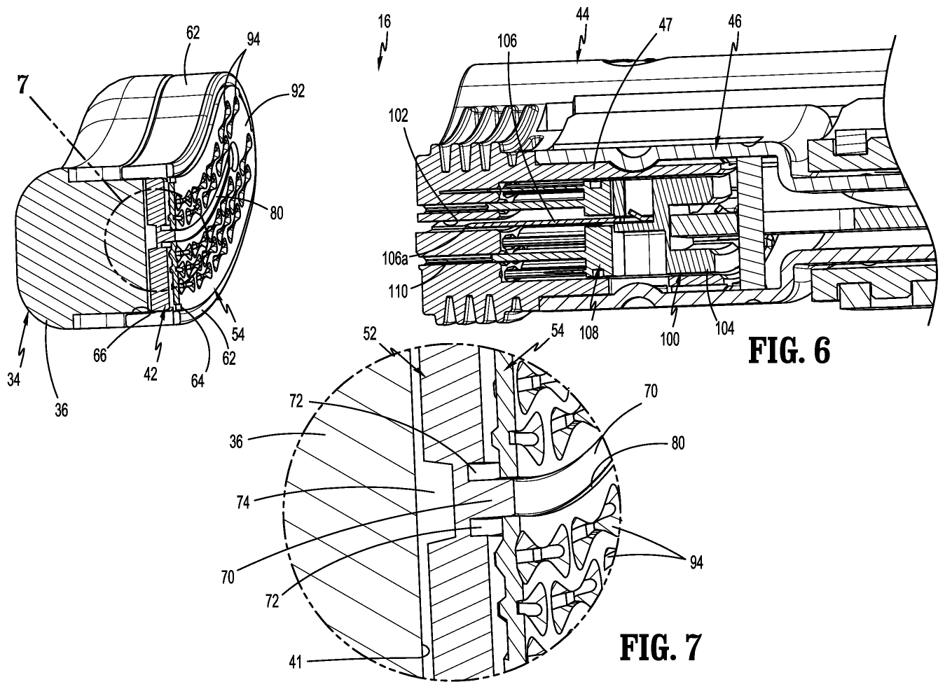

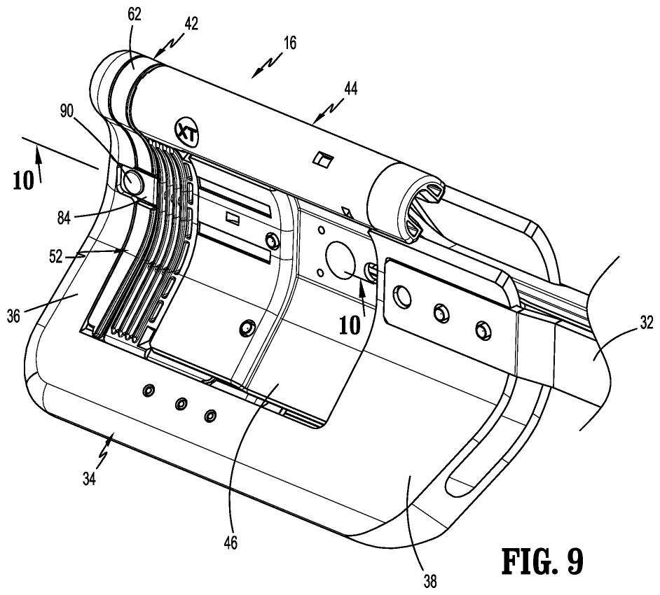

The end effector 16 includes an anvil assembly 42 and a cartridge assembly 44. The cartridge assembly 44 is removably supported on a clamp slide assembly 46 of the stapling device 10 and includes a body 47 that supports a plurality of staples 110 (FIG. 6). The clamp slide assembly 46 includes a distal portion 46a that is configured to releasably support the cartridge assembly 44. The distal portion 46a is positioned in the gap 40 and is movable between retracted and advanced positions to move the cartridge assembly 44 in relation to the anvil assembly 42 through the gap 40 between an unclamped position (FIG. 1) located adjacent to the second transverse portion 38 of the distal frame portion 34 and a clamped position (FIG. 9) located adjacent to the first transverse portion 36 of the distal frame portion 34. For a detailed description of exemplary aspects of the operation and construction of a clamp slide assembly, see the '508 Patent.

FIGS. 2-8 illustrate the anvil assembly 42 of the end effector 16 of the stapling device 10. The anvil assembly 42 is supported on the first transverse portion 36 of the distal frame portion 34 and includes a cutting plate 52 and an anvil plate 54. The cutting plate 52 and the anvil plate 54 have configurations that correspond to the configuration of the first transverse portion 36 of the distal frame portion 34. The cutting plate 52 includes a body portion 60 and a side wall 62 that extends about a periphery of the body portion 60 to define proximal and distal recesses 64 and 66 (FIG. 5) on the proximal and distal sides of the cutting plate 52. The side wall 62 defines opposed cutouts 62a. The body portion 60 includes a first end 60a positioned adjacent to the longitudinal portion 39 of the distal frame portion 34 and a second end 60b that is positioned adjacent to the free end 36a (FIG. 2) of the first transverse portion 36 of the distal frame portion 34. The distal recess 66 receives the first transverse portion 36 of the distal frame portion 34. When the cutting plate 52 is supported on the first transverse portion 36 of the distal frame portion 34, the side wall 62 of the cutting plate 52 is received within a recess 68 (FIG. 2) that is formed about the periphery of the first transverse portion 36 of the distal frame portion 34 to securely position the cutting plate 52 on the distal frame portion 34 of the stapling device 10.

The body portion 60 of the cutting plate 52 includes a central rib 70 that extends along most of the length of the cutting plate 52 within the proximal recess 64. A first relief groove 72 (FIG. 5) is formed into a proximal surface of the body portion 60 of the cutting plate 52 on each side of the central rib 70. In some aspects of the disclosure, a second relief groove 74 (FIG. 5) is formed in a distal surface of the body portion 60 of the cutting plate 52 beneath the central rib 70. The second relief groove 74 can have a width that extends across the central rib 70 and the first relief grooves 72. The body portion 60 of the cutting plate 52 also defines an alignment pin opening 76 that is positioned to receive the alignment pin (not shown) of the cartridge assembly 44 when the alignment pin is moved from its retracted to its advanced position. The '508 Patent describes the operation and construction of an exemplary alignment pin.

In aspects of the disclosure, the body portion 60 of the cutting plate 52 has a thickness that increases from first end 60a of the body portion 60 towards the second end 60b of the cutting plate 60. The change in thickness may be uniform along the length of the body portion 60 of the cutting plate 52 or, alternately may be non-uniform and selected to achieve uniform cutting of tissue as described below. In aspects of the disclosure, the first end 60a of the cutting plate 52 can have a thickness of from about 0.020 inches to about 0.040 inches and the second end 60b of the body portion 60 can have a thickness of from about 0.065 inches to about 0.090 inches. In certain aspects of the disclosure, the first end 60a of the body portion 60 of the cutting plate 52 has a thickness of about 0.029 inches and the second 60b of the body portion 60 has a thickness of about 0.077 inches.

In aspects of the disclosure, the cutting plate 52 is formed from a plastic material such as a polyether ether ketone (PEEK) material, a polyoxymethylene (POM) material, or a polyphenylsulfone material (PPSU)polypropylene, polyethylene, a thermoplastic elastomer, polyurethane, thermoplastic polyurethanes or combinations thereof. In other aspects of the disclosure, the cutting plate 76 is formed by injection molding although other methods of manufacture are envisioned. The cutting plate 76 should be formed of a material that will deform locally and structurally upon engagement with the knife 102. Local deformation of the cutting plate 76 ensures that no gap exists between the knife 102 and the surface of the cutting plate 76 to facilitate a clean cut through tissue. The structural deformation limits the firing force required to fire the stapling device 10.

FIG. 4 illustrates the anvil plate 54 of the anvil assembly 42 which includes a body 54a that defines a knife slot 80 and an alignment pin bore 82 that receives the alignment pin (not shown). The anvil plate 54 includes a first end 55a and a second end 55b and is positioned on top of the cutting plate 52 within the proximal recess 64 to sandwich the cutting plate 52 between the anvil plate 54 and the first transverse portion 36 of the distal frame portion 34. In aspects of the disclosure, the anvil plate 54 includes flanges 84 (FIG. 2) and the first transverse portion 36 of the distal frame portion 34 defines cutouts 86 (FIG. 2). The flanges 84 extend through the cutouts 62a of the cutting plate 52 and are secured within the cutouts 86 of the first transverse portion 36 of the distal frame portion 34 to secure the anvil assembly 42 to the first transverse portion 36 of the distal frame portion 34. In certain aspects of the disclosure, the flanges 84 define openings 88 that receives a rivet or pin 90 to secure the anvil plate 54 and the cutting plate 52 to the first transverse portion 36 of the distal frame portion 34. The anvil plate 54 includes a proximal staple forming surface 92 which defines staple forming pockets 94 on each side of the knife slot 80.

FIGS. 6 and 7 illustrate the end effector 16 of the stapling device 10 with the anvil assembly 42 secured to the distal transverse portion 36 of the of the distal frame portion 34 and the cartridge assembly 44 supported within the clamp slide assembly 46. In this position, the cutting plate 52 is sandwiched between the distal transverse portion 36 of the central rib 72 of the cutting plate 52 is aligned and projects partially through the knife slot 80 of the anvil plate 54. It is envisioned that the central rib 70 of the cutting plate 52 can extend entirely through the knife slot 80 of the anvil plate 54 or, in the alternative, can be recessed slightly beneath the knife slot 80 of the anvil plate 54.

FIG. 6 illustrates the cartridge assembly 44 supported within the clamp slide assembly 46 of the stapling device 10. As illustrated, the cartridge assembly 44 includes a knife assembly 100 that is supported within the body 47 of the cartridge assembly 44. The cartridge body 47 defines a knife slot 102. The knife assembly 100 includes a knife carrier 104 and a knife 106. The knife 106 includes a cutting edge 106a that is aligned with the central rib 70 of the cutting plate 52. The cartridge assembly 44 also includes a staple pusher 108 that is moved from a retracted position to an advanced position within the cartridge body 44 to eject staples 110 from the cartridge body 47 into the anvil assembly 44. When the staples 110 are driven into the anvil assembly 42, the force applied to the anvil assembly 42 causes the free end 36a of the first transverse portion of the distal frame portion 34 to bend distally in relation to the first end of the first transverse portion 36. This causes the second end 55b of the anvil plate 55 to move distally in relation to the first end of the anvil plate 54. The clamp slide assembly 46 is movable from a retracted position to an advanced position to move the cartridge assembly 44 towards the anvil assembly 42 from an open position to a clamped position.

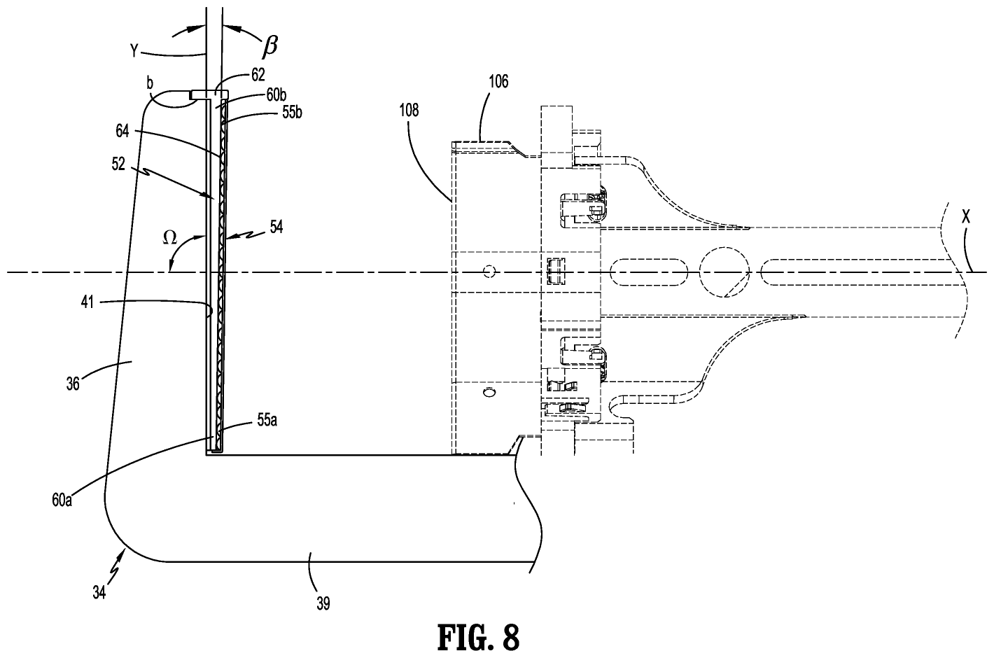

FIG. 8 illustrates a schematic side view of the distal frame portion 34 of the stapling device 10 (FIG. 1) with the knife assembly 100 shown in phantom in a retracted position. As illustrated, the planar proximal surface 41 of the first transverse portion 36 of the distal frame portion 34 defines a plane “Y” that defines an angle Ω with the longitudinal axis “X” of the elongate body 14. The angle Ω may be substantially 90 degrees. The distal surface of the cutting plate 52 rests upon the proximal surface 41 of the first transverse portion 36. Since the thickness of the cutting plate 52 increases from the first end 60a of the body portion 60 of the cutting plate 52 to the second end 60b of the cutting plate 52, the proximal surface of the body 60 of the cutting plate 52 defines an angle β with the plane “Y”. In aspects of the disclosure, β is from about 1 degree to about 3 degrees. In some aspects of the disclosure, β is about 1.70 degrees. Since the anvil plate 54 is supported atop the proximal surface of the cutting plate 52, the body 54a of the anvil plate 54 also defines angle β with the plane “Y”. As such, the second end 55b of the anvil plate 54 is angled towards the cartridge assembly 44 and is positioned proximally of the first end 55a of the anvil plate 54.

FIGS. 9-12 illustrate the end effector 16 of the stapling device 10 after the stapling device 10 (FIG. 1) is fired. Prior to firing of the stapling device 10, the clamp slide assembly 46 is moved from a retracted position to an advanced position to move the cartridge assembly 44 in relation to the anvil assembly 42 from the open position (FIG. 1) to the clamped position (FIG. 9). The '508 Patent discloses operation of an exemplary clamp slide assembly for moving a cartridge assembly of a stapling device towards an anvil assembly from the open position to a clamped position.

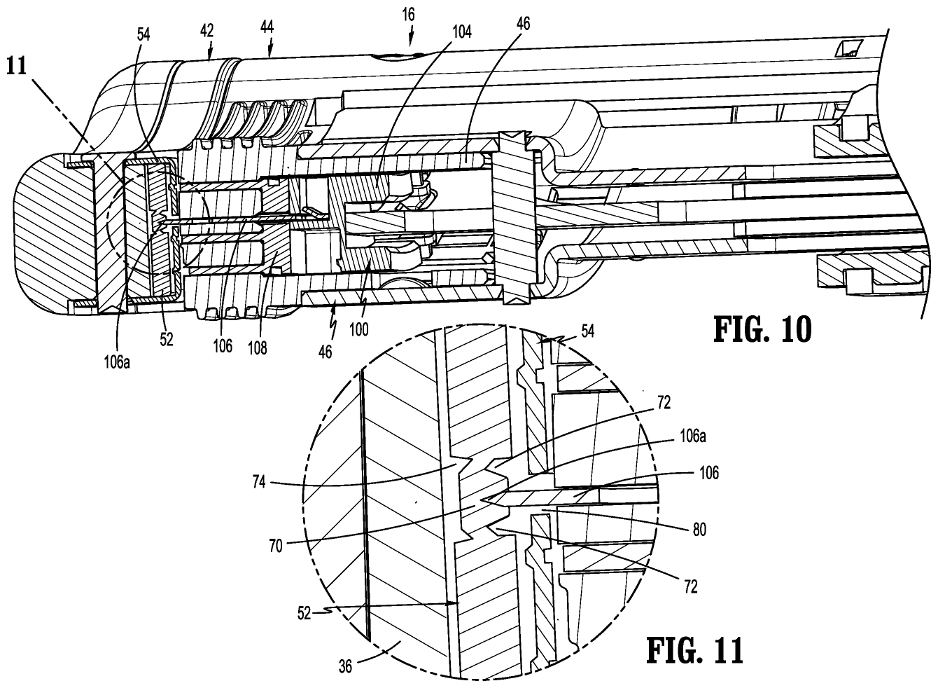

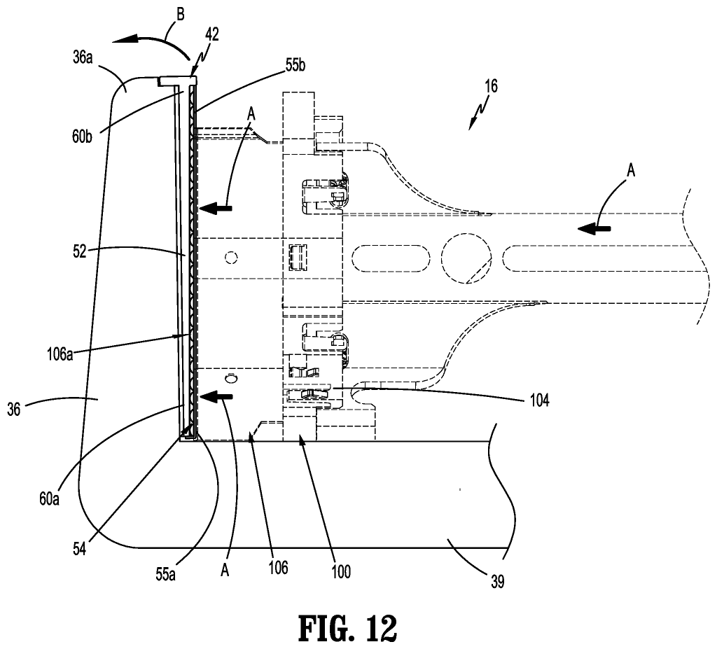

When the stapling device 10 (FIG. 1) is fired, the staple pusher 108 (FIG. 10) is advanced in the direction of arrows “A” in FIG. 12 to drive the staples 110 (FIG. 6) from the cartridge assembly 44 into the anvil assembly 52 and the knife assembly 100 (FIG. 9) is advanced in the direction of arrows “A” in FIG. 12 to advance the knife 106 through the knife slot 80 (FIG. 11) in the anvil plate 54 into the central rib 70 of the cutting plate 52 of the anvil assembly 42. Prior to firing of the stapling device 10, the anvil plate 54 is positioned as shown in FIG. 8 with the second end 55b of the anvil plate 54 angled distally towards (positioned closer to) the cartridge assembly 44. When the stapling device 10 is fired and forces are applied to the anvil assembly 42 by the staples 110 as the staples 110 are formed on the anvil plate 54, the free end 36a (FIG. 12) of the first transverse portion 36 of the distal frame portion 34 is deformed distally in the direction of arrow “B” in FIG. 12 to a position in which the first and second ends 55a and 55b of the anvil plate 54 are substantially aligned along an transverse axis to the longitudinal axis “X” of the elongate body 14 (FIG. 1) of the stapling device 10. This allows for the knife 106 to apply a cutting force that is evenly distributed along the central rib 70 of the cutting plate 52 to effect complete transection or resection of tissue (not shown) that is clamped between the anvil assembly 42 and the cartridge assembly 44.

When the knife 106 is advanced from its retracted position to its advanced position in the direction indicated by arrows “A” in FIG. 12, the cutting edge 106a of the knife 106 of the knife assembly 100 engages the central rib 70 of the cutting plate 52 and applies a force in the distal direction to the central rib 70. As described above, first and second relief grooves 72 and 74 are positioned on proximal and distal surfaces of the cutting plate 52 adjacent the central rib 70. The relief grooves allow the central rib 70 to be deformed distally into the second relief groove 74 (FIG. 11). This allows the knife 106 to move in relation to tissue (not shown) that is clamped between the anvil and cartridge assemblies 42 and 44 to ensure that the tissue (not shown) clamped between the anvil and cartridge assemblies 42 and 44 is cleanly and completely transected or resected. As noted above, the thickness of the cutting plate 52 varies from the first end 60a of the cutting plate 52 to the second end 60b of the cutting plate 52. This change in thickness of the cutting plate 52 is provided to compensate for deformation of the free end 36a of the first transverse portion 36 in the direction of arrow “B” in FIG. 12 as the stapling device 10 (FIG. 1) is fired. More specifically, the change in the thickness of the cutting plate 52 allows central rib 70 of the cutting plate to move to a position that is substantially parallel to the cutting edge 106a of the knife 106 as the stapling device 10 (FIG. 1) is fired to evenly distribute the cutting forces of the knife 106 along the central rib 70 of the cutting plate 52 as the first transverse portion 36 of the distal frame portion 34 deforms distally during firing of the stapling device 10 (FIG. 1).

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect of the disclosure may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. An end effector comprising:
a frame having a U-shaped configuration and including a first transverse portion, a second transverse portion, and a longitudinal portion interconnecting the first transverse portion and the second transverse portion, the first transverse portion being spaced from the second transverse portion to define a gap, the first transverse portion of the frame having a first end coupled to the longitudinal portion of the frame and a second free end; and
an anvil assembly supported on the first transverse portion of the frame, the anvil assembly including a cutting plate and an anvil plate, the cutting plate having a first end positioned adjacent the first end of the first transverse portion of the frame and a second end positioned adjacent the second free end of the first transverse portion of the frame, the anvil plate secured to the first transverse portion of the frame proximally of the cutting plate such that the cutting plate is sandwiched between the anvil plate and the first transverse portion, wherein the thickness of the cutting plate increases from the first end of the cutting plate to the second end of the cutting plate.

2. The end effector of claim 1, wherein the cutting plate is formed from a material selected from polyether ether ketone, polyoxymethylene, a polyphenylsulfone, polypropylene, polyethylene, a thermoplastic elastomer, polyurethane, thermoplastic polyurethanes, or combinations thereof.

3. The end effector of claim 1, wherein the cutting plate includes a proximally extending central rib and the anvil plate defines a knife slot, the central rib aligned with the knife slot.

4. The end effector of claim 3, wherein the cutting plate includes a body having a proximally facing surface, and the central rib extends proximally from the proximally facing surface.

5. The end effector of claim 4, wherein the proximally facing surface includes first relief grooves that extend along opposite sides of the central rib of the cutting plate.

6. The end effector of claim 5, wherein the cutting plate includes a distally facing surface, the distally facing surface defining a second relief groove that is aligned with the central rib.

7. The end effector of claim 6, wherein the second relief groove has a width that extends across the central rib and the first relief grooves.

8. The end effector of claim 4, wherein the cutting plate has a side wall that extends about the body, the side walls extending proximally of the body to define a proximal recess and distally of the body to define a distal recess, the first transverse portion of the frame received within the distal recess and the anvil plate received within the proximal recess.

9. The end effector of claim 1, wherein the anvil assembly is curved.

10. A surgical stapling device comprising:
a handle assembly;
a frame defining a longitudinal axis, the frame having a distal frame portion having a U-shaped configuration, the distal frame portion including a first transverse portion, a second transverse portion, and a longitudinal portion interconnecting the first transverse portion and the second transverse portion, the first transverse portion spaced from the second transverse portion to define a gap, the first transverse portion of the distal frame portion having a first end coupled to the longitudinal portion of the distal frame portion and a second free end;
a clamp slide assembly having a distal portion supported within the gap, the distal portion of the clamp slide assembly including a cartridge receiving portion;
a cartridge assembly supported on the cartridge receiving portion of the clamp slide assembly; and
an anvil assembly supported on the first transverse portion of the distal frame portion, the anvil assembly including a cutting plate and an anvil plate, the cutting plate having a first end positioned adjacent the first end of the first transverse portion of the distal frame portion and a second end positioned adjacent the second free end of the first transverse portion of the distal frame portion, the anvil plate secured to the first transverse portion of the distal frame portion proximally of the cutting plate such that the cutting plate is sandwiched between the anvil plate and the first transverse portion of the distal frame portion, wherein the thickness of the cutting plate increases from the first end of the cutting plate to the second end of the cutting plate.

11. The surgical stapling device of claim 10, wherein the cutting plate includes a proximally extending central rib and the anvil plate defines a knife slot, the central rib aligned with the knife slot.

12. The surgical stapling device of claim 11, wherein the cutting plate includes a body having a proximally facing surface, and the central rib extends proximally from the proximally facing surface.

13. The surgical stapling device of claim 12, wherein the proximal surface includes first relief grooves that extend along opposite sides of the central rib of the cutting plate.

14. The surgical stapling device of claim 13, wherein the cutting plate includes a distal surface, the distal surface defining a second relief groove that is aligned with the central rib.

15. The surgical stapling device of claim 14, wherein the second relief groove has a width that extends across the central rib and the first relief grooves.

16. The surgical stapling device of claim 12, wherein the cutting plate has a side wall that extends about the body, the side walls extending proximally of the body to define a proximal recess and distally of the body to define a distal recess, the first transverse portion of the frame received within the distal recess and the anvil plate received within the proximal recess.

17. The surgical stapling device of claim 10, wherein the anvil assembly is curved along an axis transverse to the longitudinal axis of the frame.

18. An anvil assembly comprising:
a cutting plate and an anvil plate, the cutting plate having a first end and a second end, the anvil plate positioned in juxtaposed alignment with the cutting plate, wherein the thickness of the cutting plate increases from the first end of the cutting plate to the second end of the cutting plate.

19. The anvil assembly of claim 18, wherein the cutting plate includes a proximally extending central rib and the anvil plate defines a knife slot, the central rib aligned with the knife slot.

20. The anvil assembly of claim 18, wherein the cutting plate includes a body having a proximally facing surface and a distally facing surface, the central rib extending proximally from the proximally facing surface, the proximally facing surface defining first relief grooves that extend along opposite sides of the central rib of the cutting plate and the distally facing surface defining a second relief groove that is aligned with the central rib.
